(19) United States
(12) Patent Application Publication
(10) Pub. No.: US 2015/0335776 A1
(43) Pub. Date: Nov. 26, 2015

(54) ADJUSTABLE CARRIER

(71) Applicant: SYNERGY HEALTH PLC, Swindon (GB)

(72) Inventors: Daniel Turner, San Diego, CA (US); Andrew McLean, Miami, FL (US); Christopher Bock, San Diego, CA (US)

(21) Appl. No.: 14/719,179

(22) Filed: May 21, 2015

Related U.S. Application Data
(60) Provisional application No. 62/002,096, filed on May 22, 2014.

(51) Int. Cl. A61L 2/26; B65G 17/20; B65G 17/32; A61L 2/08

(52) U.S. Cl. CPC A61L 2/26; A61L 2/087; B65G 17/20; B65G 17/32

(57) ABSTRACT

An adjustable carrier for a sterilisation system having a platform configured to provide support for products to be sterilised. The adjustable carrier includes an adjustment mechanism configured to adjust a length of the platform in an intended direction of travel of the carrier.

### CROSS-REFERENCE TO RELATED APPLICATIONS

This application claims benefit of priority under 35 U.S.C. §119(e) of U.S. Ser. No. 62/002,096, filed May 22, 2014, the entire contents of which is incorporated herein in its entirety.

### FIELD OF THE INVENTION

The present invention relates to an adjustable carrier for a sterilisation system, a conveyance arrangement for a sterilisation system, and to a method of adjusting an adjustable carrier.

### BACKGROUND OF TILE INVENTION

Sterilisation systems for medical devices and equipment utilise a variety of different systems, such as gamma ray or electron beam systems.

These systems typically place the product onto a carrier which is then moved in front of the radiation via a conveyance arrangement. These allow for the loading and unloading of the products to be done at a location remote from the sterilisation process. The speed of travel around the conveyance arrangement is determined by the dose required for the product to be sterilised.

The carriers used currently have a fixed length along the direction of travel of the carrier. The product boxes are placed on the carrier, however it is rare that the entire area of the carrier is utilised and this results in a loss of throughput for the sterilisation system.

The present invention seeks to overcome or at least mitigate the problems of the prior art.

### SUMMARY OF THE INVENTION

A first aspect of the invention provides an adjustable carrier for a sterilisation system, the adjustable carrier comprising a platform configured to provide support for products to be sterilised and an adjustment mechanism configured to adjust a length of the platform in an intended direction of travel of the carrier.

Advantageously, this allows for the minimisation of unused space within the carrier and allows the throughput of products through the sterilisation system to be increased.

In one embodiment, the adjustment mechanism is manually adjustable.

Advantageously, this allows for an easy method of adjusting the carrier and may enhance reliability.

In one embodiment, the adjustment mechanism further comprises a drive interface for powered adjustment of the carrier.

Advantageously, this allows for an easy method of adjusting the carrier which may also be quicker than manual adjustment. This powered adjustment may enable adjustment to be carried out at a location away from radiation to avoid the radiation exposure in the system causing the electronics to fail.

In one embodiment, the adjustment mechanism comprises a plurality of detents at predetermined spacing.

Advantageously, this allows for easy monitoring of the length of adjustment in-situ.

In one embodiment, the adjustable carrier further comprises walls extending substantially vertically from the edges of the platform.

Advantageously, this supports the products to be sterilised, reducing the risk of them being dislodged.

In one embodiment, the platform comprises a front and a rear section.

In one embodiment, the adjustment mechanism is arranged to move the front and rear sections by equal amounts with respect to a centre of the carrier.

In one embodiment, the carrier is configured to travel on a conveyance arrangement of a sterilisation system.

In one embodiment, the carrier further comprises drive formations to engage a drive of the conveyance arrangement.

In one embodiment, the carrier is configured to be supported from underneath.

In one embodiment, the carrier comprises a plurality of mounting arrangements to enable to the carrier to be suspended from the conveyance arrangement.

In one embodiment, the adjustment mechanism comprises a mechanism for converting a rotary input into a linear adjustment of the adjustable carrier.

In one embodiment, the adjustment mechanism comprises a worm drive.

A second aspect of the present invention provides a conveyance arrangement for a sterilisation system comprising a plurality of adjustable carriers according to the first aspect of the present invention, the arrangement comprising, a loading station for loading the products to be sterilised onto the carriers; a sterilisation station remote from the loading station for the products to be sterilised; an unloading station for removing the products from the carriers; and a conveyor for transferring carriers between the loading station, the sterilisation station and the unloading station.

In one embodiment, the conveyor is configured to travel at a predetermined speed along the sterilisation station relating to the product to be sterilised.

In one embodiment, the conveyor is a continuous loop.

In one embodiment, the conveyance arrangement further comprises a length adjustment station.

In one embodiment, the conveyance arrangement further comprises a control system configured to set the length of the adjustable carrier at the length adjustment station.

The control system may additionally control the conveyance arrangement to ensure appropriate spacing between carriers.

A third aspect of the present invention provides a method of adjusting an adjustable carrier for a sterilisation system, comprising the steps of: determining the length of the product to be placed thereon; determining the adjustment required to minimise the empty area of the adjustable carrier; and adjusting the length of the carrier to substantially match the length of the product to be sterilised.

A fourth aspect of the invention provides a method of sterilisation of a product to be sterilised, comprising the steps of: placing the product to be sterilised onto an adjustable carrier; driving the adjustable carrier along the conveyance arrangement; passing the product to be sterilised through a sterilisation station sterilise the product; and removing the product from the adjustable carrier, wherein before or after placing the product to be sterilised on the adjustable carrier, the length of the carrier is compared with the length of the product, and the length of the carrier is adjusted, if required, to substantially match the length of the product to be sterilised.

### BRIEF DESCRIPTION OF THE DRAWINGS

Embodiments of the invention will now be described with reference to the accompanying drawings, in which:

FIG. 1 is a schematic plan view of a sterilisation system;

FIGS. 2A and 2B are plan views of adjustable carriers in an extended and retracted configuration respectively;

FIG. 3 is a side of an adjustable carrier in a retracted configuration;

FIG. 4 is a side view of the adjustable carrier of FIG. 3 in an extended configuration;

FIGS. 5A and 5B are side and bottom views respectively of the adjustment portion of the adjustable carrier of FIG. 3;

FIG. 6 is an exploded plan view of the guide portion of the adjustable carrier of FIG. 3;

FIG. 7 is a plan view of the guide portion of the adjustable carrier of FIG. 3; and

FIG. 8 is a cross sectional side view of the guide portion of FIG. 7 along the line A-A.

### DETAILED DESCRIPTION OF EMBODIMENT(S)

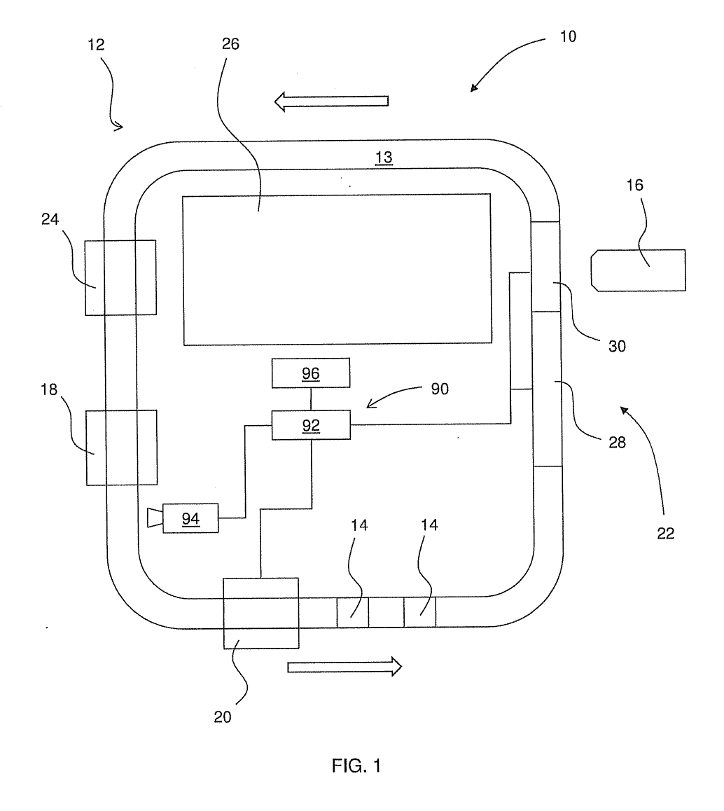

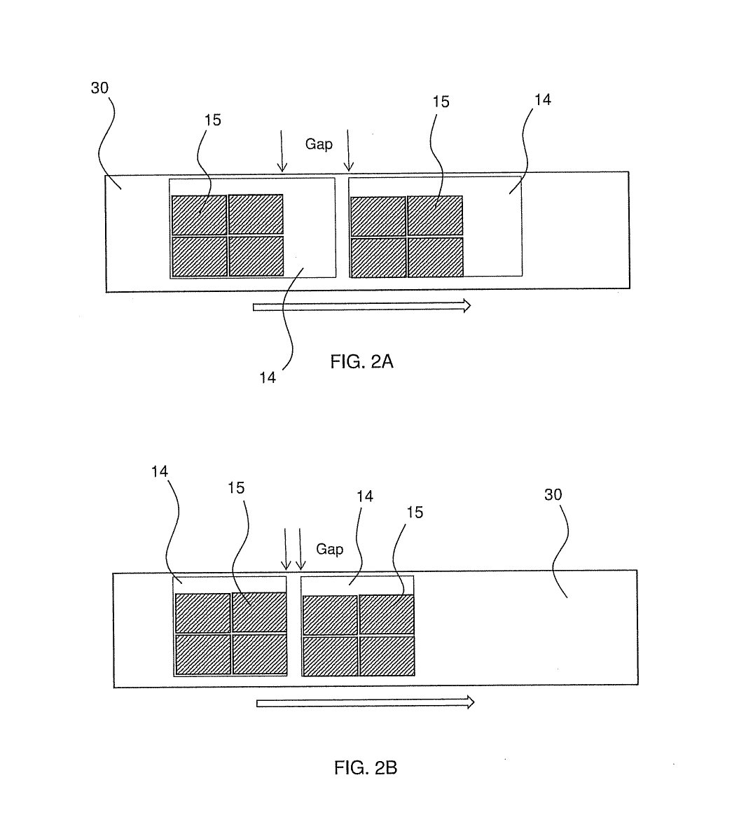

Referring firstly to FIG. 1, an electron beam sterilisation system of an embodiment of the present invention is indicated generally at 10. The sterilisation system 10 includes a conveyance arrangement 12, upon which a series of adjustable carriers 14 are placed and are configured to travel around the conveyance arrangement, and an electron emitter 16 configured to emit an electron beam. The conveyance arrangement 12 comprises a conveyor 13 configured to transport the adjustable carriers 14 in front of the electron emitter 16 whilst carrying one or more product boxes 15 (FIGS. 2A and 2B) in order to expose the products within to electron beam radiation. Typical products which may be sterilised in this way include medical and laboratory products such as petri-dishes, intravenous sets and general labware.

The conveyance arrangement 12 includes a loading station 18, an adjusting station 20, a sterilisation station 22 and an unloading station 24, where the sterilisation station is located remote from the others. This arrangement ensures that an individual is not exposed to the electron beam radiation while loading/unloading products onto/off the carrier or when adjusting the length of the carrier 14. The sterilisation station 22 comprises a shield 26 (e.g. a concrete mass), a closing conveyor 28, the electron emitter 16 and a process table 30 where the process table is located between the concrete shield and the electron emitter.

In this embodiment, the carriers 14 travel anti-clockwise around the sterilisation system 10 as indicated in FIG. 1. The carriers 14 travel from the loading station 18 to the adjustment station 20 and then to the sterilisation station 22 before finally arriving at the unloading station 24, where the product is removed from the carrier 14 and thus the sterilisation system 10, after being successfully sterilised. It will be appreciated that in other embodiments, the drive direction of the conveyor 13 and layout of the conveyance arrangement 12 may be altered as required.

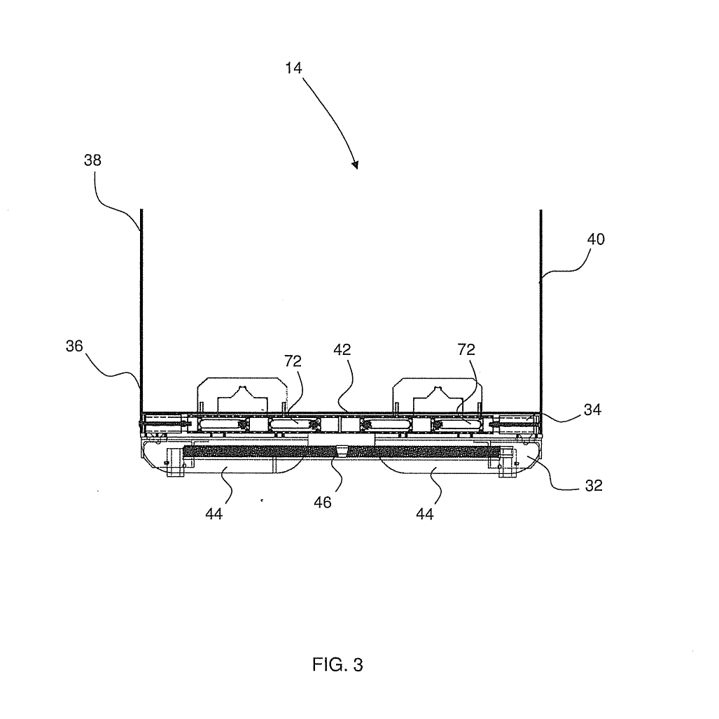

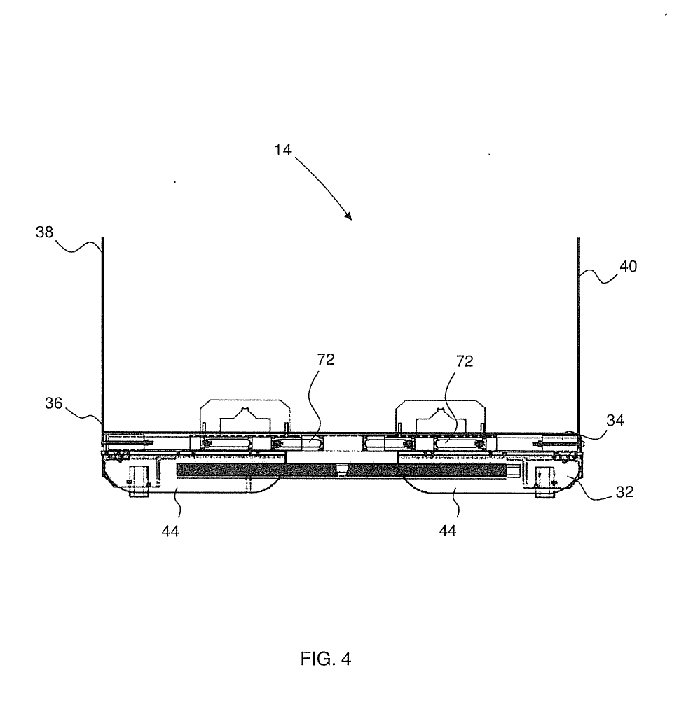

Referring to FIGS. 3 and 4, the adjustable carrier 14 is illustrated in more detail. In this embodiment, the adjustable carrier 14 is formed from three separate portions, an adjustment portion 32 configured to travel along the conveyance arrangement 12, a guide portion 34, and a platform portion 36. The guide portion 34 is supported by the adjustment portion 32 and is secured to and substantially covers the adjustment portion. The platform portion 36 is secured to the front and rear ends of the guide portion 34 and adjustment portion 32. The platform portion 36 comprises two end walls 38, 40 which extend upwardly from the front and rear faces of the guide portion 34 and a platform surface 42 which is placed over the guide portion 34 to substantially cover it and provide a smooth flat surface for the product boxes 15 to be placed upon. In certain embodiments a further guide portion (not shown) similar to guide portion 34 may extend between the top edges of the end walls 36, 40 to provide a more rigid carrier structure. In other embodiments, one or more of the adjustment portion 32, guide portion 34 and platform portion may be combined/integrated.

The end walls 38, 40 support the product boxes 15 to inhibit them from being dislodged during transport around the conveyance arrangement 12. The platform portion 36 of the carrier 14 has no side walls to enable the electron beam to pass through to the products without unnecessary obstructions to absorb radiation from the electron emitter 16.

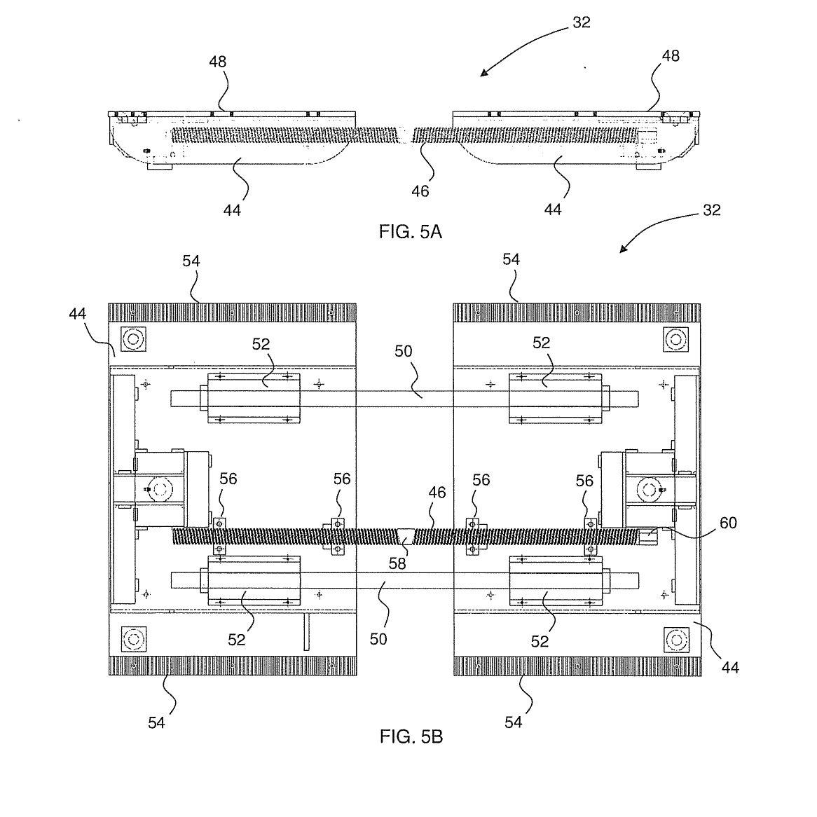

With respect to FIGS. 5A and 5B, the adjustment portion 32 of the adjustable carrier 14 is illustrated in more detail. The adjustment portion 32 consists of two carrier bases 44 substantially rectangular in plan view, which are joined by an adjustment mechanism 46. The carrier bases 44 include a substantially flat upper surface 48 upon which the guide portion 34 of the adjustable carrier 14 is mounted.

In addition to being joined by the adjustment mechanism 46, the carrier bases 44 are attached via two elongate members 50 located proximate the side edges of the base and which extend substantially parallel to the longitudinal axis of the adjustment portion 32. The underside of the carrier bases 44 each have a slide bush arrangement 52 with a bore configured to allow the elongate members 50 to slide through during expansion and contraction of the adjustment portion 32. This arrangement enables the two carrier bases 44 to stay aligned during expansion and contraction of the adjustment portion 32 and also provide a limit to the expansion of the adjustment portion.

The carrier bases 44 include toothed tracks 54 which extend along the underside of the carrier base proximate to the side edges to allow the carrier bases to engage with a corresponding configuration (not shown) on the conveyor 13 in order to propel the carrier 14 along said conveyor.

In this embodiment, the adjustment mechanism 46 is in the form of a single screw with two screw threads of opposite handedness. The screw 46 is attached to the underside of each carrier base 44 via a fixing arrangement 56 with internal threaded bores on each base. The screw 46 comprises a central linking segment 58 located between the carrier bases 42. This orientation allows for the expansion and contraction to be equal for both carrier bases 44 upon rotation of the screw 46 resulting in faster adjustment of the carrier 14. The screw 46 has an interface 60 at one end, a hex head in this embodiment, for a drive to be releasably connected thereto to adjust the length of the carrier 14.

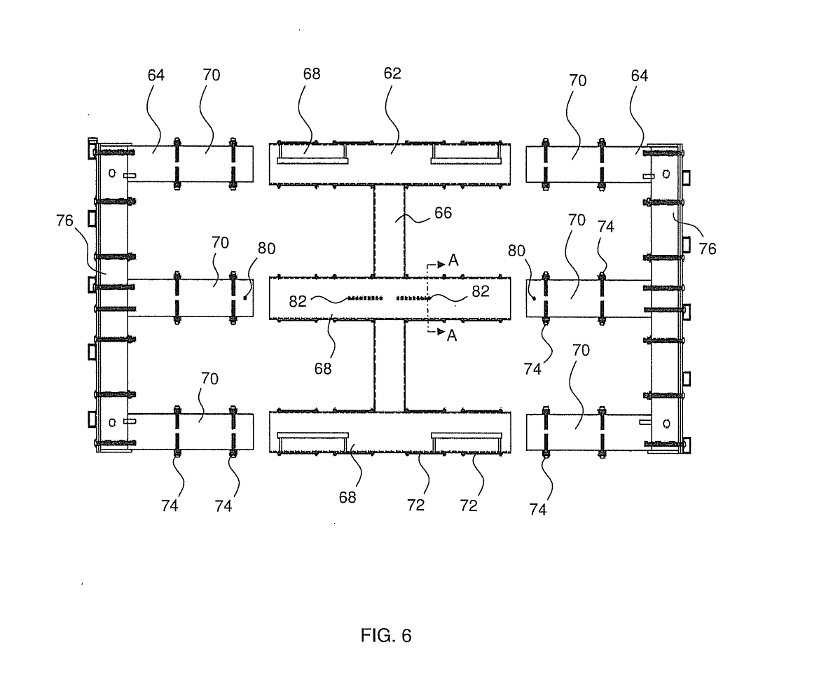

Referring to FIGS. 6, 7 and 8, the guide portion 34 of the adjustable carrier 14 is illustrated in more detail. The guide portion 34 includes a central section 62 and two end sections 64.

The central section 62 has a cross member 66 which extends side-to-side (i.e. perpendicularly to an end-to-end longitudinal axis of the guide portion 34) and further includes three members 68, one located at each end and one in the centre of the member 66, which extend longitudinally in both directions from the cross member 66. The members 68 define a recess at both ends to allow a protruding member 70 of the end sections 64 to slide into.

Each of the members 68 include a series of elongate slots 72 (as illustrated in FIGS. 3 and 4) in each side surface thereof configured to receive a fastener 74 when the central section 62 is assembled with the end sections 64. The fasteners mount a guide collar 88 which extend around the fastener and enable smoother movement of the fastener 74 within the slot.

Each end section 64 comprises a cross member 76 which is substantially parallel to the cross member 66 of the central section 62. The three protruding members 70 of the end sections 64 extend perpendicularly to the cross member 76 and are configured to slide into the recess in the corresponding members 68 of the central section 62. Each protruding member 70 comprises threaded bores to receive the fasteners. When the fastener 74 is inserted through the bore and into the elongate slot 72 the elongate slots limit the movement of the end section 64 with respect to the central section 62.

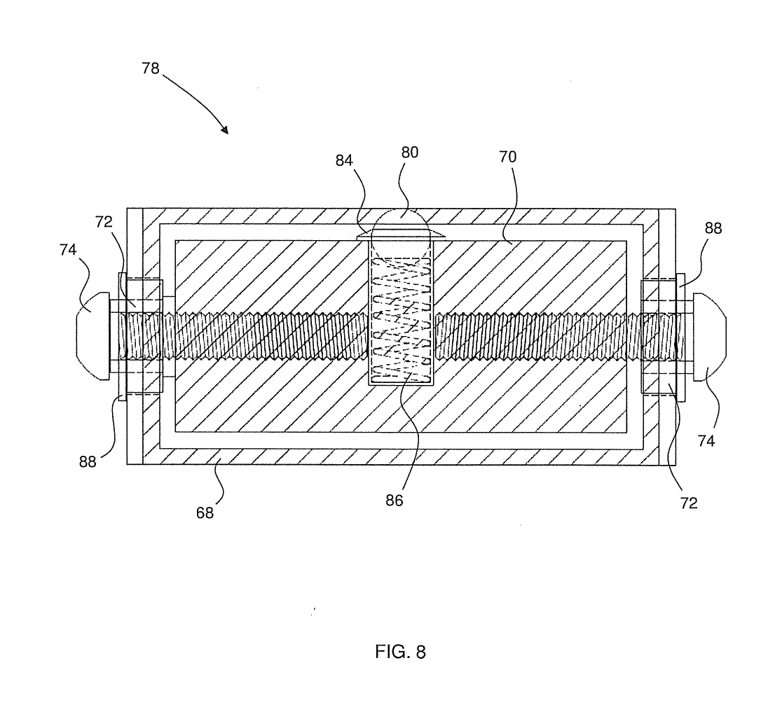

The guide portion 34 also includes two detent arrangements 78 as illustrated in FIG. 8. The detent arrangements 78 are located on the central of the three members 70 and include a bore with a ball 80 and a spring 86 located within the bore. The springs 86 are configured to urge the respective balls 80 against a retainer 84 which holds the ball in place. The detent arrangements 78 are configured to allow the ball 80 to protrude through a hole in the retainer 84 and the upper surface of the central member 70.

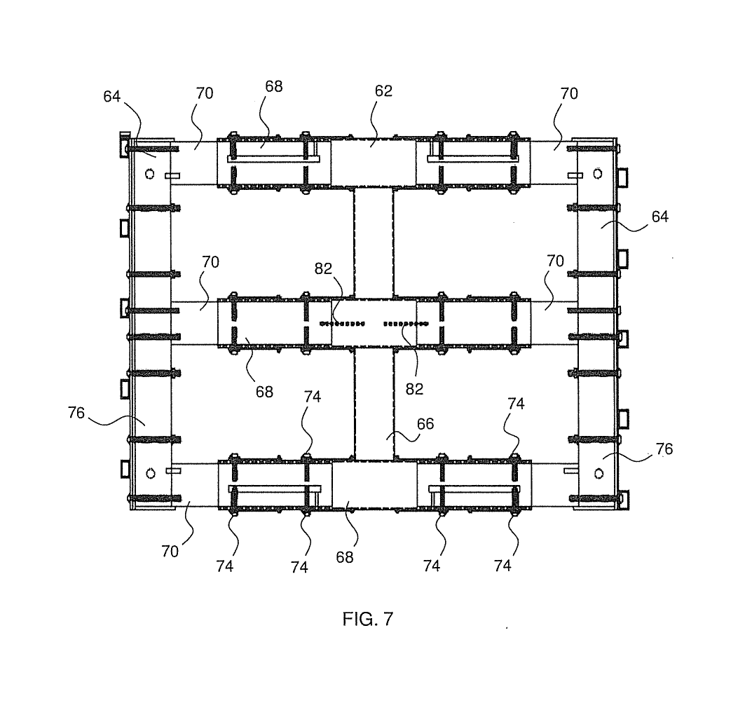

The central member 68 of the central section 62 of the guide portion 34 includes a series of circular holes 82 in the upper surface thereof which extend parallel to the longitudinal axis of the guide portion 34 (as illustrated in FIGS. 6 and 7).

When assembled, the end section 64 is inserted into the recesses of the central section 62. The balls 80 locate within, and extend into, the circular holes 82 of the central member under the influence of the spring 86, temporarily fixing the positions of the central 62 and end 64 sections of the guide portion 34 with respect to each other. The spacing between each of the holes 82 correspond to a length of extension or contraction of the carrier 14, and the spacing can be altered to correspond to the desired incremental adjustment. In this embodiment, the typical range of adjustment can adjust the length of the carrier 14 from approx. 40″-48″ (around 101 to 122 cm) in 1″ (2.5 cm) increments.

The adjustment station 20 comprises a socket (not shown) configured to engage the hex interface 60 of the carriers 14 and a servomotor and resolver (not shown) to drive the socket and monitor the rotation thereof. The adjustment station may further comprise a deployment mechanism (not shown) to move the socket into alignment with the interface as a carrier approaches on the conveyor, should adjustment thereof be required.

The sterilisation system 10 further includes a control system 90 which includes a controller 92 (a suitable microprocessor controller in this embodiment), a reader 94, and a database 96. The packaging of the product to be sterilised has an identifier such as an RFID tag or a barcode and the reader 94 reads the identifier and then references the database 96 to determine the dose required by that product and the product dimensions. The controller 92 is able to adjust the speed of the conveyance arrangement 12 over the process table 30 accordingly.

The controller 92 is further capable of adjusting the speed of the closing conveyor 28 to close up gaps between adjacent carriers 14 prior to their passing on to the process table 30. Further, the controller 92 may control operation of the servomotor and deployment mechanism to adjust the length of a carrier 14 to match that of the product boxes 15 placed on the carrier 14. In other embodiments, adjusting the length of the adjustable carrier 14 may be done manually.

In operation, the product boxes 15 are placed on the adjustable carriers 14 at the loading station 18 and are read by the reader 94 and identified by the controller 92 utilising the database 96. The adjustable carriers 14 then travel along the conveyor 13 to the adjustment station 20.

As shown in FIGS. 2A and 2B, the size and orientation of the product boxes 15 may result in less than optimal packing within the carrier 14. This can result in gaps being present at the rear or front of the carrier 14 at which the electron beam is not being effectively utilised for sterilisation. The conveyance arrangement 12 is unable to change the travel speed over the process table 30 to accommodate for these gaps and so potential processing time is lost in these regions.

Thus, whilst at the adjustment station 20 the length of the carrier 14 along the direction of travel along the conveyance arrangement 12 may be automatically adjusted if the controller 92 determines that its current length does not match that of the product boxes 15 to minimise the unused area within the carrier. This in turn minimises the time that the electron beam is not being effectively used, and results in increased the throughput of the system 10.

In other embodiments adjustment may be semi-automated. For example the controller may visually and/or audibly indicate to an operator when adjustment of a carrier 14 is needed, they may engage the socket on to the interface 60 and the controller 92 may then control drive to the socket to correctly adjust the length. In an alternative embodiment, the adjustment of the carrier 14 may be carried out prior to the carrier being placed on the conveyance arrangement 12 at a location upstream or downstream of the loading station 18.

Although it is possible to incorporate a motor with an electronic control system into the carriers 14 this is not a preferred configuration as the radiation from the electron beam tends to damage the electronics.

Gaps are present between adjacent carriers 14 whilst travelling around the conveyor 13, where the gaps are sufficiently wide enough to enable the loading of products onto the carrier and the adjustment of the carrier length without adjacent carriers colliding. These wide gaps are reduced when travelling over the closing conveyor 28 (again under the control of the control system 90), such that the carriers 14 travel with minimum separation over the process table 30, as illustrated in FIGS. 2A and 2B. In this embodiment, the conveyor 13 is configured to carry the product boxes 15 around the system at a speed of around 0.2 meters per second. Whilst travelling over the closing conveyor 28 the speed of travel is reduced so that the speed of travel along the process table 30 is typically between 0.015 and 0.020 meters per second.

The conveyance arrangement 12 is configured so that the carriers 14 and product boxes 15 pass between the electron emitter 16 and the shield 26, exposing the product to electron beam radiation.

Following sterilisation, the speed of travel of the carriers 14 increases again which increases separation between adjacent carriers, and travels to the unloading station 24 where the sterilised products are removed from the carrier 14.

Although the invention has been described above with reference to one or more preferred embodiments, it will be appreciated that various changes or modifications may be made without departing from the scope of the invention as defined in the appended claims.

In alternative embodiments, the sterilisation system may comprise a second electron emitter may be located on the opposing side of the product to be sterilised to the first electron emitter. This two sided irradiation allows for a greater thickness of product to be processed, where the dose applied to the product is the sum of the first and second electron emitters.

In alternative arrangements, the sterilisation system may comprise different conveyance arrangements. The conveyance arrangement may be configured to suspend the carriers from above.

Adjustment of the adjustable carriers may alternatively be activated through a range of linear actuators such as hydraulic, pneumatic, or electromagnetic actuators or may comprise alternative gear mechanisms such as a rack and pinion system. In other embodiments adjustment may occur between two parts only, rather than between a central part and two ends.

In alternative embodiments, the sterilisation system may comprise a gamma ray source in place of the electron emitter.

What is claimed is:

1. An adjustable carrier for a sterilisation system, the adjustable carrier comprising a platform configured to provide support for products to be sterilised and an adjustment mechanism configured to adjust a length of the platform in an intended direction of travel of the carrier.

2. An adjustable carrier according to claim 1, wherein the adjustment mechanism is manually adjustable.

3. An adjustable carrier according to claim 1, wherein the adjustment mechanism further comprises a drive interface for powered adjustment of the carrier.

4. An adjustable carrier according to claim 1, wherein the adjustment mechanism comprises a plurality of detents at predetermined spacing.

5. An adjustable carrier according to claim 1, further comprising walls extending substantially vertically from the edges of the platform.

6. An adjustable carrier according claim 1, wherein the platform comprises a front and a rear section.

7. An adjustable carrier according to claim 6, wherein the adjustment mechanism is arranged to move the front and rear sections by equal amounts with respect to a centre of the carrier.

8. An adjustable carrier according claim 1, wherein the carrier is configured to travel on a conveyance arrangement of a sterilisation system.

9. An adjustable carrier according to claim 8, further comprising drive formations to engage a drive of the conveyance arrangement.

10. An adjustable carrier according to claim 9, wherein the carrier is configured to be supported from underneath.

11. An adjustable carrier according to claim 9, further comprising a plurality of mounting arrangements to enable to the carrier to be suspended from the conveyance arrangement.

12. An adjustable carrier according to claim 1, wherein the adjustment mechanism comprises a mechanism for converting a rotary input into a linear adjustment of the adjustable carrier.

13. An adjustable carrier according to claim 12, wherein the adjustment mechanism comprises a worm drive.

14. A conveyance arrangement for a sterilisation system comprising a plurality of adjustable carriers according to any preceding claim, the arrangement comprising, a loading station for loading the products to be sterilised onto the carriers; a sterilisation station remote from the loading station for the products to be sterilised; an unloading station for removing the products from the carriers; and a conveyor for transferring carriers between the loading station, the sterilisation station and the unloading station.

15. A conveyance arrangement according to claim 14, wherein the conveyor is configured to travel at a predetermined speed along the sterilisation station relating to the product to be sterilised.

16. A conveyance arrangement according to claim 14, wherein the conveyor is a continuous loop.

17. A conveyance arrangement according to claim 14, further comprising a length adjustment station.

18. A conveyance arrangement according to claim 17, further comprising a control system configured to set the length of the adjustable carrier at the length adjustment station.

19. A conveyance arrangement according to claim 14, further comprising a control system to control the conveyance arrangement to ensure appropriate spacing between carriers.

20. A method of adjusting an adjustable carrier for a sterilisation system, comprising the steps of:
a) determining the length of the product to be placed thereon;
b) determining the adjustment required to minimise the empty area of the adjustable carrier; and
c) adjusting the length of the carrier to substantially match the length of the product to be sterilised.
